(19) United States
(12) Patent Application Publication
(10) Pub. No.: US 2024/0131305 A1
(43) Pub. Date: Apr. 25, 2024

(54) VASODILATOR INTRODUCER SHEATH

(71) Applicant: Medtronic, Inc., Minneapolis, MN (US)

(72) Inventor: Niall F. Duffy, Tuam (IE)

(21) Appl. No.: 18/487,054

(22) Filed: Oct. 13, 2023

Related U.S. Application Data
(60) Provisional application No. 63/380,734, filed on Oct. 24, 2022.

(51) Int. Cl. A61M 25/09; A61M 25/00

(52) U.S. Cl. CPC A61M 25/09; A61M 25/0026; A61M 2025/004; A61M 2025/0681; A61M 2025/09133; A61M 2202/0007; A61M 2210/12

(57) ABSTRACT

An introducer includes an introducer hub and a vasodilating drug-delivery introducer sheath coupled to the hub and extending distally therefrom. The introducer sheath may include a primary lumen and infusion lumens for delivering a therapeutic agent such as a vasodilator through ports in the introducer sheath or may include an outer porous layer for the therapeutic agent to be delivered to the vessel, or may include at least one drug-coated outer layer for the therapeutic agent to be released from the drug-coated outer layer.

### CROSS-REFERENCE TO RELATED APPLICATIONS

This application claims the benefit of U.S. Provisional Application No. 63/380,734, filed Oct. 24, 2022, the contents of which are incorporated by reference herein in their entirety.

### FIELD OF THE INVENTION

The present invention relates to an introducer for providing percutaneous access to a patient's vasculature for a transcatheter device delivery system, and, more particularly to an introducer having sheath configured to deliver a vasodilating drug to minimize damage to the patient's vessel during removal of the introducer from the vessel.

### BACKGROUND

An introducer is used to provide percutaneous access to the vascular system of a patient and functions to permit the introduction and positioning of various minimally invasive delivery devices within the patient's vasculature. In general, making the smallest incision is the most desirable and leads to less complications, less trauma, and improved patient outcomes. In some cases, such as introducers used for access for transcatheter heart valve prosthesis delivery devices, the introducer sheaths have larger diameters to accommodate the transcatheter heart valve prosthesis delivery device. In such cases, problems can occur when inserting and removing the introducer from the patient's vessel. The tight fit of the introducer in the vessel can result in the vessel being damaged, or in extreme circumstances, stripped from the body. Thus, there is a need for introducers that are able to deliver vessel expanding drugs to dilate the vessel of the patient to accommodate removal of such introducers from within the body.

### BRIEF SUMMARY OF THE INVENTION

In accordance with a first example hereof, an introducer includes an introducer hub and an introducer sheath defining a primary lumen configured to receive a medical device therethrough, wherein a vasodilating therapeutic agent is configured to be released from the introducer sheath to expand a vessel into which the introducer sheath is inserted

In a second example, in the introducer according to any of the previous or subsequent examples herein, the introducer sheath further includes an infusion lumen adjacent to the primary lumen and a plurality of ports extending from an exterior surface of the introducer sheath to the infusion lumen, wherein the introducer sheath is configured to receive the vasodilating therapeutic agent in the infusion lumen and to release the vasodilating drug through the plurality of ports.

In a third example, in the introducer according to any of the previous or subsequent examples herein, the introducer sheath includes exactly one infusion lumen.

In a fourth example, in the introducer according to any of the previous or subsequent examples herein, the introducer sheath includes exactly two infusion lumens.

In a fifth example, in the introducer according to any of the previous or subsequent examples herein, the introducer sheath includes exactly three infusion lumens.

In a sixth example, in the introducer according to any of the previous or subsequent examples herein,

In a seventh example, in the introducer according to any of the previous or subsequent examples herein, the infusion lumen is an annular lumen surrounding the primary lumen.

In an eighth example, in the introducer according to any of the previous or subsequent examples herein, the plurality of ports extend from adjacent a distal end of the introducer hub to adjacent a distal end of the introducer sheath.

In a ninth example, in the introducer according to any of the previous or subsequent examples herein, the plurality of ports extend along only a distal portion of the introducer sheath.

In a tenth example, in the introducer according to any of the previous or subsequent examples herein, the plurality of ports extend along only a proximal portion of the introducer sheath.

In an eleventh example, in the introducer according to any of the previous or subsequent examples herein, the plurality of ports increase in size in a distal direction along the longitudinal length of the introducer sheath.

In a twelfth example, in the introducer according to any of the previous or subsequent examples herein, the plurality of ports are uniform in size.

In a thirteenth example, in the introducer according to any of the previous or subsequent examples herein, the plurality of ports have a substantially circular or oval-shape cross-section.

In a fourteenth example, in the introducer according to any of the previous or subsequent examples herein, the introducer sheath includes a tube having the primary lumen disposed therethrough and an outer porous layer covering an exterior surface of the tube.

In a fifteenth example, in the introducer according to any of the previous or subsequent examples herein, the outer porous layer comprises a copolymers of styrene-divinylbenzene or a sintered porous plastic.

In a sixteenth example, in the introducer according to any of the previous or subsequent examples herein, the outer porous layer extends from a proximal end of the tube to a distal end of the tube.

In a seventeenth example, in the introducer according to any of the previous or subsequent examples herein, the outer porous layer extends over only a proximal portion of the introducer sheath.

In an eighteenth example, in the introducer according to any of the previous or subsequent examples herein, the introducer sheath comprises a tube having the primary lumen disposed therethrough and at least one drug-coated layer disposed on an exterior surface of the tube, wherein the at least one drug-coated layer includes the vasodilating therapeutic agent.

In a nineteenth example, in the introducer according to any of the previous or subsequent examples herein, the at least one drug-coated layer is configured to elute the vasodilating therapeutic agent upon contact with bodily fluids in the vessel.

In a twentieth example, in the introducer according to any of the previous or subsequent examples herein, the at least one drug-coated layer comprises a plurality of drug-coating layers configured to elute the vasodilating therapeutic agent over time.

In a twenty-first example, in the introducer according to any of the previous or subsequent examples herein, the plurality of drug-coated layers comprises exactly three drug-coated layers.

In a twenty-second example, in the introducer according to any of the previous or subsequent examples herein, a first coated outer layer releases a drug over a first period of time, a second coated outer layer releases a drug over a second period of time that occurs after the first period of time, and a third coated outer layer releases a drug over a third period of time that occurs after the second period of time.

In a twenty-third example, in the introducer according to any of the previous or subsequent examples herein, the first period of time, the second period of time, and the third period of time overlap with one another.

In a twenty-fourth example, in the introducer according to any of the previous or subsequent examples herein, the first period of time, the second period of time, and the third period of time do not overlap with one another.

In a twenty-fifth example, in the introducer according to any of the previous or subsequent examples herein, the at least one drug-coated layer extends over from a distal end of the introducer to a distal end of the tube.

In a twenty-sixth example, in the introducer according to any of the previous or subsequent examples herein, the at least one drug-coated layer extends over only a proximal portion of the tube.

In a twenty-seventh example, in the introducer according to any of the previous or subsequent examples herein, the at least one drug-coated layer extends over only a distal portion of the tube.

In a twenty-eighth example, a method includes inserting an introducer into a vessel of a patient such that an introducer sheath of the introducer is disposed within the vessel and an introducer hub of the introducer is disposed outside of the patient, advancing a deice distally through the introducer sheath, introduced a vasodilating therapeutic agent within the vessel through the introducer such that the vasodilating therapeutic agent dilates the vessel, and removing the introducer sheath from the vessel after the vessel is dilated.

In a twenty-ninth example, in a method according to any of the previous or subsequent examples herein, the method further includes removing the device from the vessel through the introducer sheath prior to removing the introducer from the vessel.

In a thirtieth example, in a method according to any of the previous or subsequent examples herein, the vasodilating therapeutic agent is introduced prior to removing the device from the vessel.

In a thirty-first example, in a method according to any of the previous or subsequent examples herein, the vasodilating therapeutic agent is introduced after removing the device from the vessel.

In a thirty-second example, in a method according to any of the previous or subsequent examples herein, the introducer comprises any one of the introducers of examples 1 to 27.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A depicts an introducer according to embodiments hereof.

FIG. 1B depicts a dilator according to embodiments hereof.

FIG. 1C depicts an introducer dilator assembly according to embodiments hereof.

FIG. 1D depicts an introducer dilator assembly illustrated closer to scale according to embodiments hereof.

FIGS. 2A-2B depict portions of the introducer of FIG. 1A according to embodiments hereof.

FIGS. 3A-3I depict various embodiments of introducer sheaths according to embodiments hereof.

FIG. 4 depicts an introducer including an outer porous layer according to embodiments hereof.

FIG. 5A depicts an introducer including at least one drug-coated outer layer according to embodiments hereof.

FIG. 5B depicts an exploded view of an introducer sheath including at least one drug-coated outer layer according to embodiments hereof.

FIG. 5C depicts a time vs. elution rate graph of the introducer sheath with at least one drug-coated outer layer according to embodiments hereof.

FIG. 6 is a block diagram that shows a method of using the introducer dilator assembly according to embodiments hereof.

FIG. 7 depicts a step in the method of FIG. 6, depicting a guidewire positioned in the vessel of a patient according to embodiments hereof.

FIG. 8 depicts a step in the method of FIG. 6, depicting the introducer dilator assembly in the vessel of a patient according to embodiments hereof.

FIG. 9 depicts a step in the method of FIG. 6, depicting the dilator being removed from the vessel of a patient according to embodiments hereof.

FIG. 10 depicts a step in the method of FIG. 6, depicting dilation of the vessel according to embodiments hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, an introducer. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. It should be understood that use of the term “about” also includes the specifically recited number of value.

The terms “proximal” and “distal” herein when used with respect to a delivery system and/or an introducer are used with reference to the clinician using the devices. Therefore, “proximal” and “proximally” mean in the direction toward the clinician, and “distal” and “distally” mean in the direction away from the clinician.

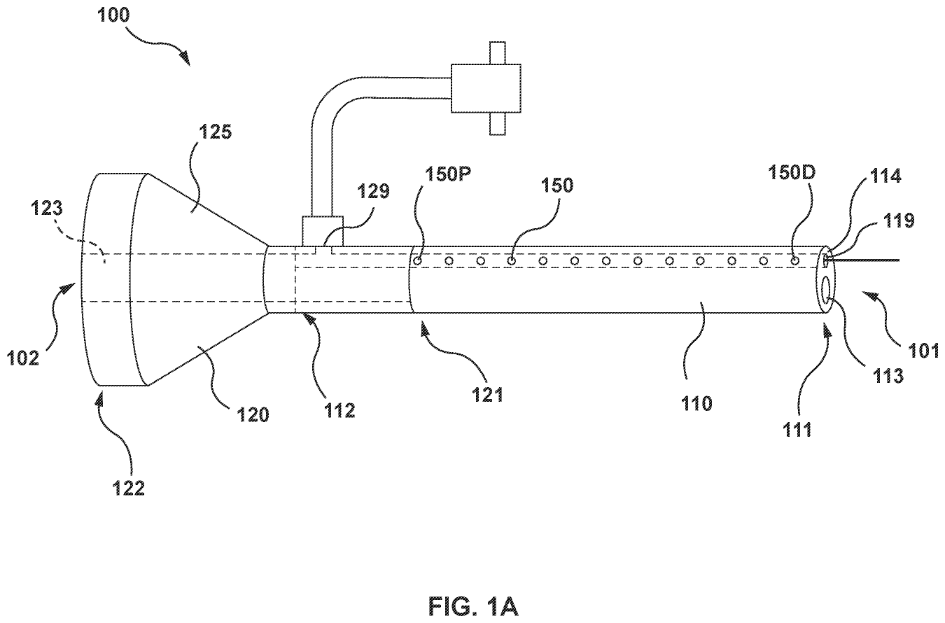

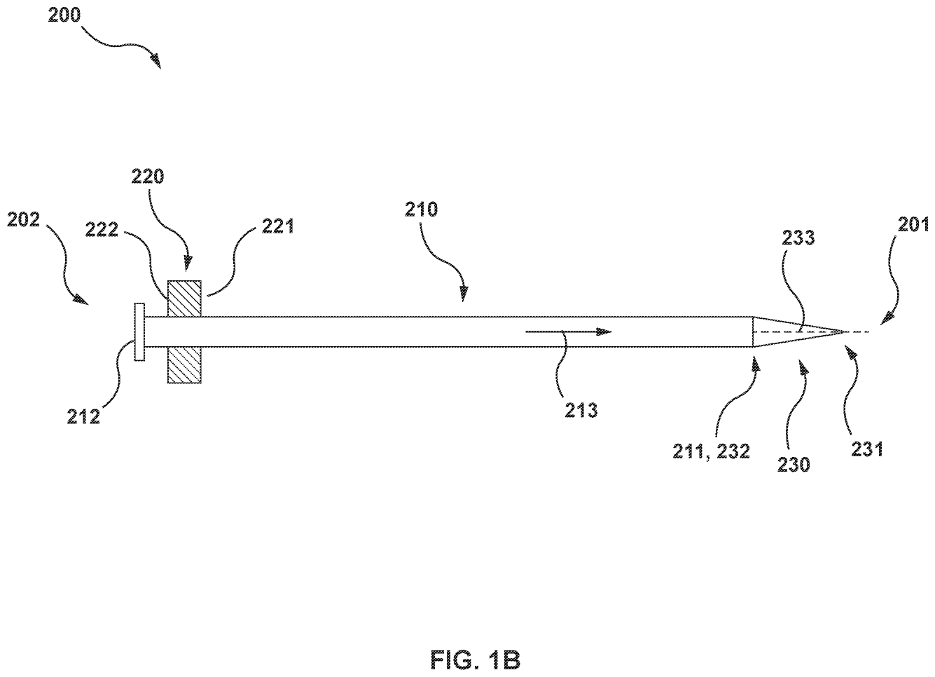

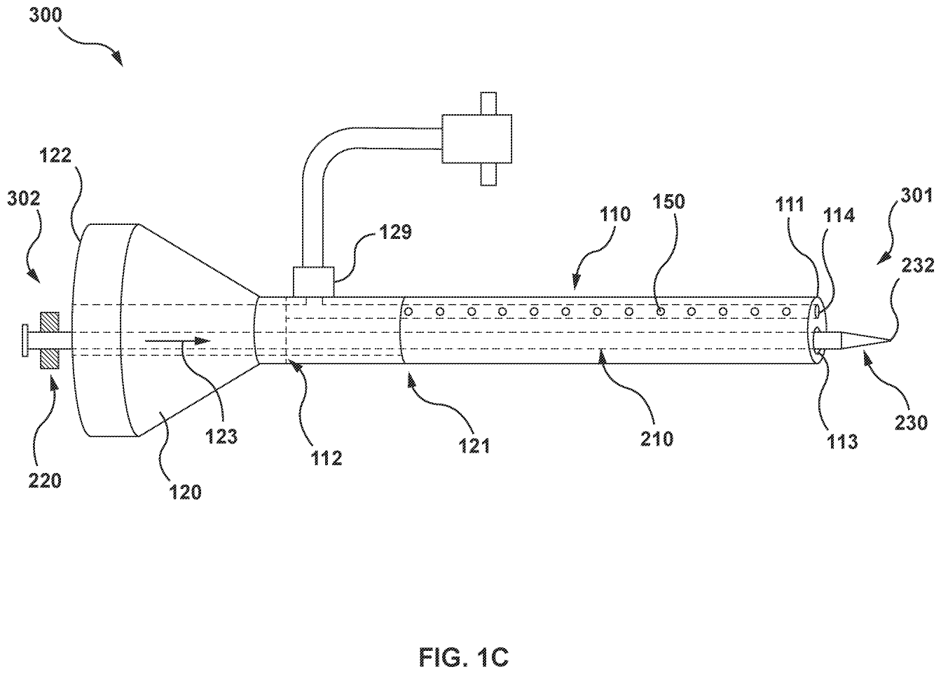

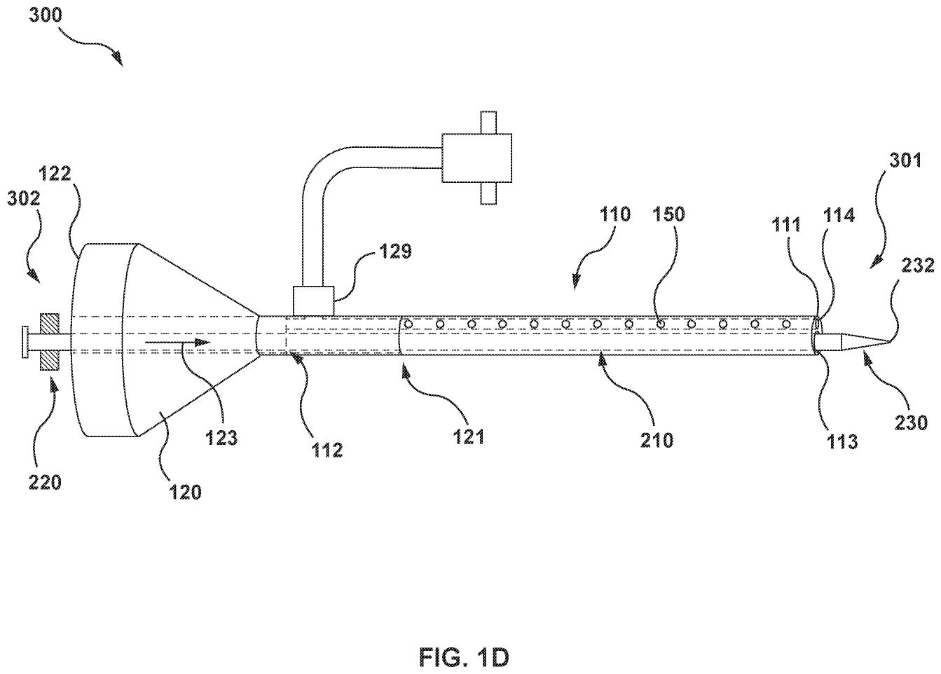

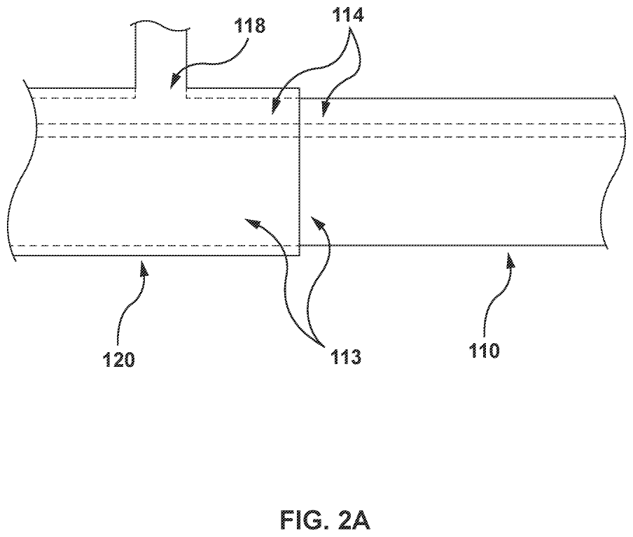

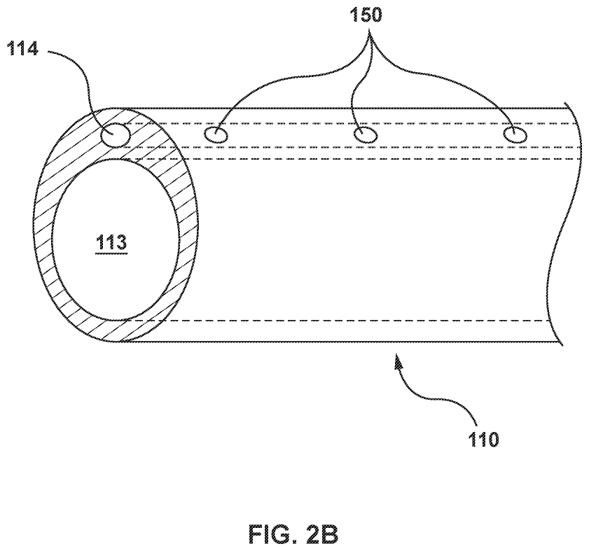

FIG. 1A schematically illustrates an introducer 100 according to embodiments herein. The introducer 100 includes a distal end 101, a proximal end 102, an introducer sheath 110, and an introducer hub 120. The introducer sheath 110 defines a primary lumen 113 extending from a proximal end 112 to a distal end 111 of the introducer sheath 110. The introducer sheath 100 further includes an infusion lumen 114, as shown in FIGS. 1A and 1C, and described further below. Although the infusion lumen 114 is shown to one side of the primary lumen 113, such as in FIG. 2B, this is not meant to be limiting, and the infusion lumen 114 may instead be, for example, an annular lumen. The infusion lumen 114 in some examples may extend from the proximal end 112 to the distal end 111 of the introducer sheath 110, but as explained below, the infusion lumen 114 may not extend to the distal end 111. In the embodiment shown in FIGS. 1A-1C, the infusion lumen 114 is closed at the proximal end 112 of the introducer sheath 110 and includes an exit port 119 at the distal end 111 of the introducer sheath 110. However, this is not meant to be limiting and the distal end of the infusion lumen 114 may instead be closed. The introducer sheath 110 may be a thin, flexible plastic tube configured to allow passage of a medical device such as a delivery catheter therethrough. In some embodiments, the introducer sheath 110 may be configured to expand from a first, unexpanded state to a second, expanded state in response to the device being advanced therethrough.

In the embodiment of FIGS. 1A-1C, the introducer sheath 110 includes openings or ports 150 extending from an exterior surface 115 of the introducer sheath 110 to the infusion lumen 114. The ports 150 enable fluid, such as a drug, injected into the infusion lumen 114 to escape the infusion lumen 114 through the ports 150 to surrounding tissue, as explained in more detail below. In the embodiment of FIGS. 1A-1C, the ports 150 a distributed along the length of the introducer sheath 110 from a proximal-most port 150P adjacent the distal end 121 of the introducer hub 120 to a distal-most port 150D adjacent the distal end 111 of the introducer sheath 110. The ports 150 are shown as generally circular, but this is not mean to be limiting, and other shapes may also be used. Further, the ports 150 are shown as generally the same size, but this is not meant to be limiting. For example, and not by way of limitation, the ports 150 may increase or decrease in size from the proximal-most port 150P to the distal-most port 150D. In other embodiments, the ports 150 may change shape along the length of the introducer sheath 110. The size, shape, and distribution of the ports 150 along the length of the introducer sheath 110 may be varied to change the elution profile of the introducer 100. Further, these size, shape, and distribution variations may apply to different embodiments described below. In an embodiment, the ports 150 are each in the range of about 0.1 to 2.0 mm in diameter, or dimensions such that the area of each port 150 is about the same as a circular port with a diameter of about 0.1 to 2.0 mm.

The introducer hub 120 includes a proximal end 122, a distal end 121, and an inner lumen or passageway 123 extending from the proximal end 122 to the distal end 121 of the introducer hub 120. The proximal end 112 of the introducer sheath 110 is coupled to the introducer hub 120 within the passageway 123 of the introducer hub 120 and extends distally from the distal end 121 of the introducer hub. The distal end 111 of the introducer sheath 110 defines the distal end 101 of the introducer 100 and the proximal end 122 of the introducer hub 120 defines the proximal end 102 of the introducer 100. The proximal end 112 of the introducer sheath 110 may be coupled to the distal end 121 of the introducer hub 120 by fusion, adhesives, mechanical clamping, an interference fit, or other means known to those skilled in the art, or any combination thereof. In embodiments, the introducer sheath 110 may be a polymer or nylon material, or other materials known to those skilled in the art suitable for the purposes described herein, or any combination thereof.

The passageway 123 of the introducer hub 120 is sized and shaped to accommodate a dilator shaft 210 of a dilator 200 therethrough, as explained in more detail below. The exterior surface 125 of the introducer hub 120 tapers in a distal direction such that the distal end 121 of the introducer hub 120 has a first diameter smaller than a second diameter of the proximal end 122 of the introducer hub 120, as shown in FIG. 1A. In some embodiments, the first diameter of the introducer hub 120 is about 4 to 10 mm and the second diameter of the introducer hub 120 is about 10 to 30 mm. However, this is not meant to be limiting, as the introducer hub 120 can include one constant diameter that does not taper in any direction. In embodiments, the introducer hub 120 includes a side port 129 which may be utilized for infusing vasodilating drugs or other fluids into the infusion lumen 114 of the introducer sheath 110, as shown in FIG. 1A and described in further detail below.

FIG. 1B shows a dilator 200 having a distal end 201, a proximal end 202, a dilator shaft 210, a dilator hub 220 and a dilator tip 230. The dilator shaft 210 includes a distal end 211, a proximal end 212, and a central lumen 213 extending from the distal end 211 to the proximal end 212. The proximal end 212 of the dilator shaft 210 is coupled to the dilator hub 220 and extends distally therefrom. The dilator tip 230 is coupled to the distal end 211 of the dilator shaft 210. The dilator tip 230 is a conical element that includes a distal end 231 and a proximal end 232 and a passageway 233 extending therethrough. The dilator tip 230 extends distally from the distal end 221 of the dilator shaft 220. The distal end 231 of the dilator tip 230 defines the distal end 201 of the dilator 200 and the proximal end 212 of the dilator shaft 210 defines the proximal end 202 of the dilator 200. The dilator tip 230 may taper in a distal direction, such that the distal end 231 of the dilator tip 230 has a first diameter that is smaller than a second diameter of the proximal end 232 of the dilator tip 230.

FIG. 1C shows the introducer 100 and the dilator 200 combined to create an introducer dilator assembly 300 to be used in medical procedures. As shown in FIG. 1C, the dilator shaft 210 extends through the proximal end 122 of the introducer hub 120, through the passageway 123 of the introducer hub 120, and through the central lumen 113 of the introducer sheath 110 of the introducer 100. The dilator 200 is advanced such that the dilator tip 230 protrudes distally past the distal end 111 of the introducer sheath 110. The dilator hub 220 is located proximal to the introducer hub 120.

The introducer dilator assembly 300, as shown and described with respect to FIG. 1C and FIGS. 8-9 are not drawn to scale. Those of skill in the art will recognize and understand that the introducer sheath 110 of the introducer 100 and the dilator shaft 210 of the dilator 200 are much closer in size. The size of the infusion lumen(s) 114 of the introducer sheath 110, as depicted in the drawings, is enlarged for clarity purposes. Thus, the introducer sheath 110 is shown larger than the dilator shaft 210. FIG. 1D shows an introducer dilator assembly 300, illustrated closer to scale, to depict the actual size of an introducer sheath 110 of an introducer 100 relative to a dilator shaft 210 of a dilator 200 for clarity purposes.

FIG. 2A shows a close up view of a portion of the introducer 100 of FIG. 1A. As can be seen in FIG. 2A and as described above, the proximal end 112 of the introducer sheath 110 is disposed within the passageway 123 of the introducer hub 120. The introducer sheath may include an opening 118 aligned with the port 128 of the introducer hub 102. The opening 118 is in fluid communication with the infusion lumen 114 of the introducer sheath 110. Thus, fluid such as a drug, injected into the infusion lumen 114 of the introducer sheath 110 through the port 129 of the introducer hub 120 and the opening 118 in the introducer sheath 110. However, this is not meant to be limiting, and other ways to inject fluid into the infusion lumen 114 of the introducer sheath 110, such as a port at the proximal end thereof.

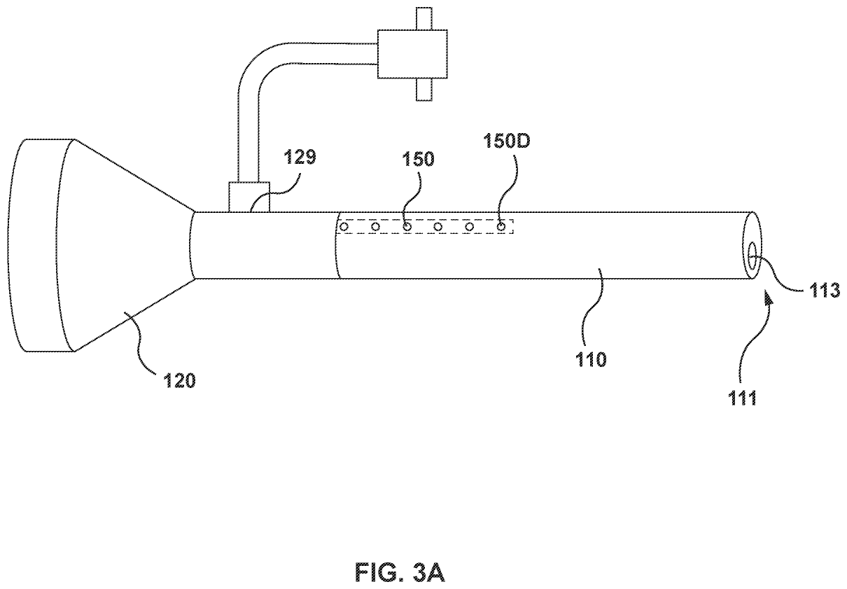

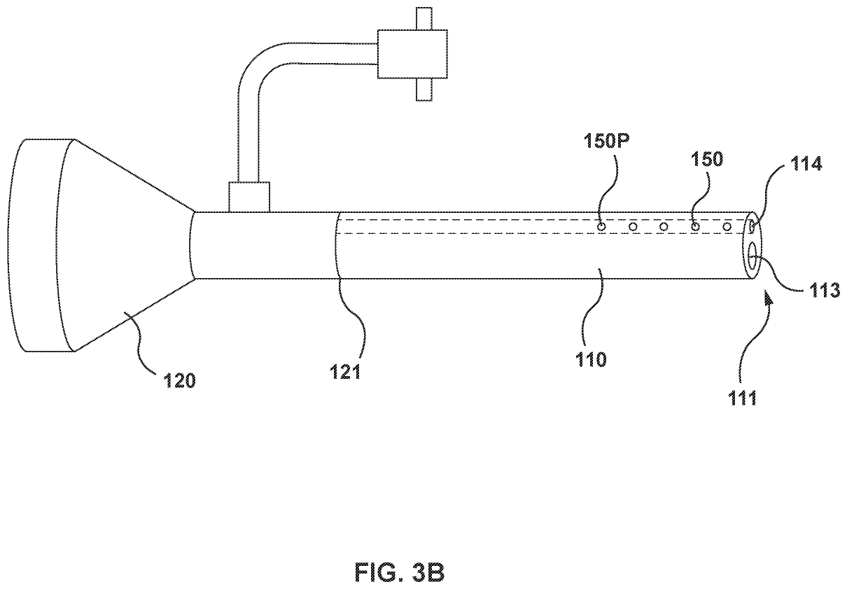

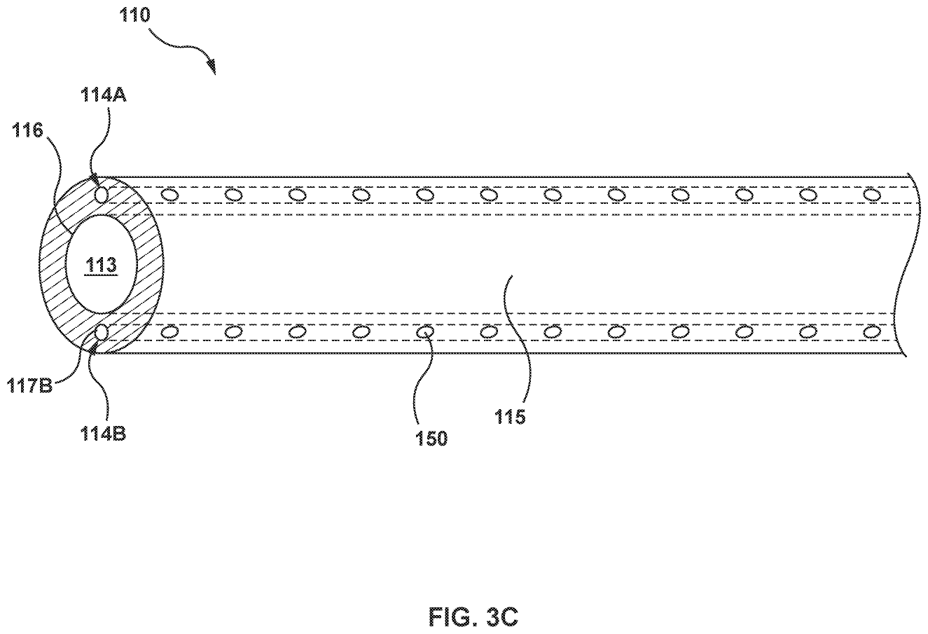

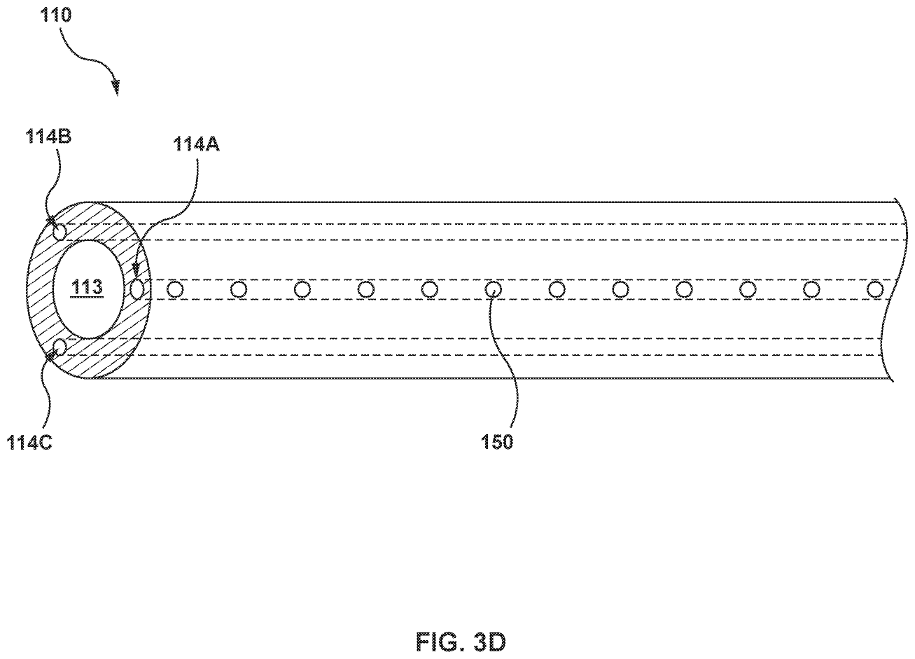

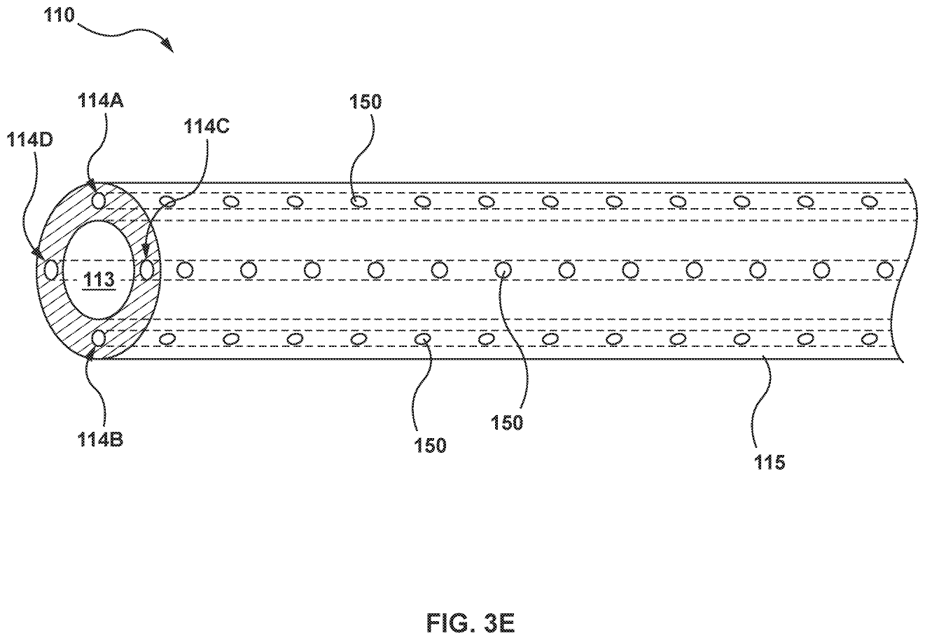

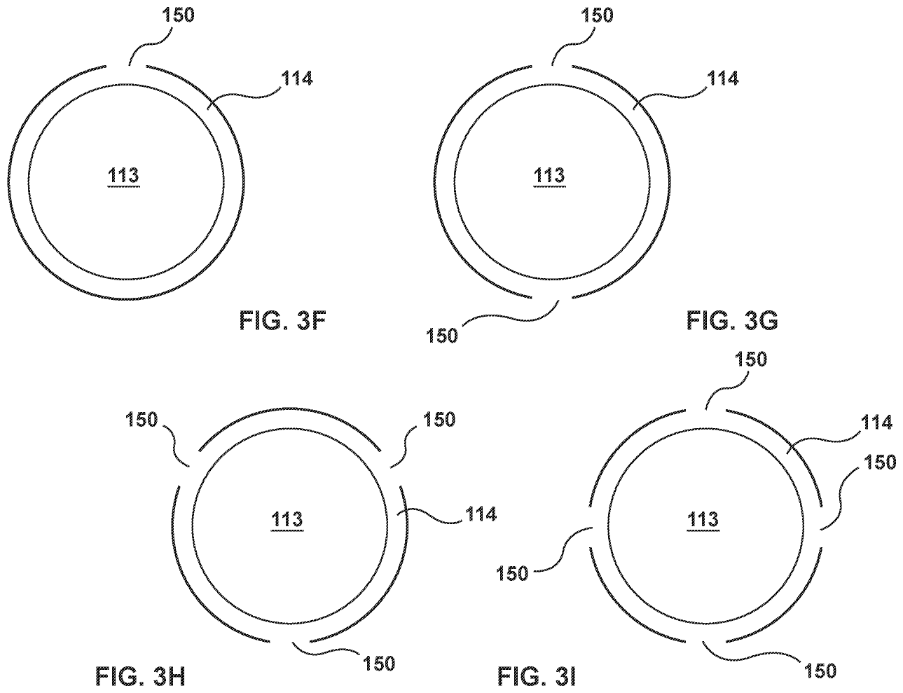

FIG. 3A shows an exemplary introducer 100 with an introducer sheath 110 according to embodiments hereof. In the embodiment of FIG. 3A, the infusion lumen 114 of the introducer sheath 110 terminates proximal of the distal end of the introducer sheath 110. Therefore, the introducer sheath 110 only includes ports 150 of the proximal portion of the introducer sheath 110. In other words, the distal-most port 150D of the introducer sheath 110 of FIG. 3A is spaced proximally from the distal end 111 of the introducer sheath 110. In an embodiment, the distal-most port 150D is located proximal of the mid-point between the distal end 121 of the introducer hub 120 and the distal end 111 of the introducer sheath 110.

In another exemplary embodiment shown in FIG. 3B, the ports 150 are disposed only on a distal portion of the introducer sheath 110. In other words, the proximal-most port 150P of the introducer sheath 110 of FIG. 3B is spaced distally from the distal end 121 of the introducer hub 120. In an embodiment, the distal-most port 150D is located proximal of the mid-point between the distal end 121 of the introducer hub 120 and the distal end 111 of the introducer sheath 110.

In the embodiments described above, the infusion lumen 114 and the ports 150 are disposed to one side of the primary lumen 113. However, as noted above, this is not meant to be limiting, and other arrangements may also be used. For example, and not by way of limitation, FIG. 3C shows an embodiment with two infusion lumens 114A, 114B disposed 180° apart from each other on opposite sides of the primary lumen 113, FIG. 3D shows an embodiment with three infusion lumens 114A, 114B, and 114C disposed evenly (about 60° apart) around the primary lumen 113, and FIG. 3E shows an embodiment with four infusion lumens 114A, 114B, 114C, and 114D disposed around the primary lumen 113 (about 90° apart) around the primary lumen 113. Each of the infusion lumens of the embodiments shown include ports 150 for enabling fluid from within the lumen to escape to the surrounding tissue, as described below. Additionally, more infusion lumens 114 or different arrangements of the infusion lumens 114 may be utilized. Further, the different arrangements of infusion lumens 114 may be used with any of the different arrangements of ports 150 described above.

As explained briefly above, instead of the arrangements described above, the infusion lumen 114 may be an annular lumen extending partially or completely around the primary lumen 113, as shown in FIGS. 3F-3I. Further, ports 150 may be included around the perimeter of the annular infusion lumen 114 in any arrangement. For example, and not by way of limitation, along at least part of the length of the introducer sheath 110, there may be a single port 150 (FIG. 3F), two ports 150 disposed 180° apart from each other on opposite sides of the primary lumen 113 (FIG. 3G), three ports 150 disposed evenly (about 60° apart) around the perimeter of the annular infusion lumen 114 (FIG. 3H), or four ports 150 disposed evenly (about 90° apart) around the perimeter of the annular infusion lumen 114 (FIG. 3I). This is not meant to be limiting and more ports may be included. Further, the arrangement and location of the ports 150 may vary along the length of the introducer sheath 110. For example, and not by way of limitation, the location of the ports 150 along the length of the introducer sheath 110 may change such that the ports 150 are in a spiral pattern. In another example, and not by way of limitation, there may be more or fewer ports 150, and/or different sized and/or shaped ports 150, towards the distal end of the introducer sheath 110.

As explained above, in use, a therapeutic agent such as a vasodilator is introduced into the infusion lumen(s) 114 through the port 129 in the hub and the opening 118 in the introducer sheath 110. However, this is not meant to be limiting and other locations and ways to inject fluid into the infusion lumen(s) 114 may be used. In embodiments disclosed herein, the fluid is a therapeutic agent, and in particular, a vasodilator. For example, and not by way of limitation, the vasodilator may be selected from the group consisting of cyclandelate, isoxsuprine, papaverine, dipyridamole, isosorbide dinitrate, phentolamine, nicotinyl alcohol, co-dergocrine, nicotinic acid, glycerol trinitrate, pentaerythritol tetranitrate, xanthanol, and other vasodilators known to those skilled in the art.

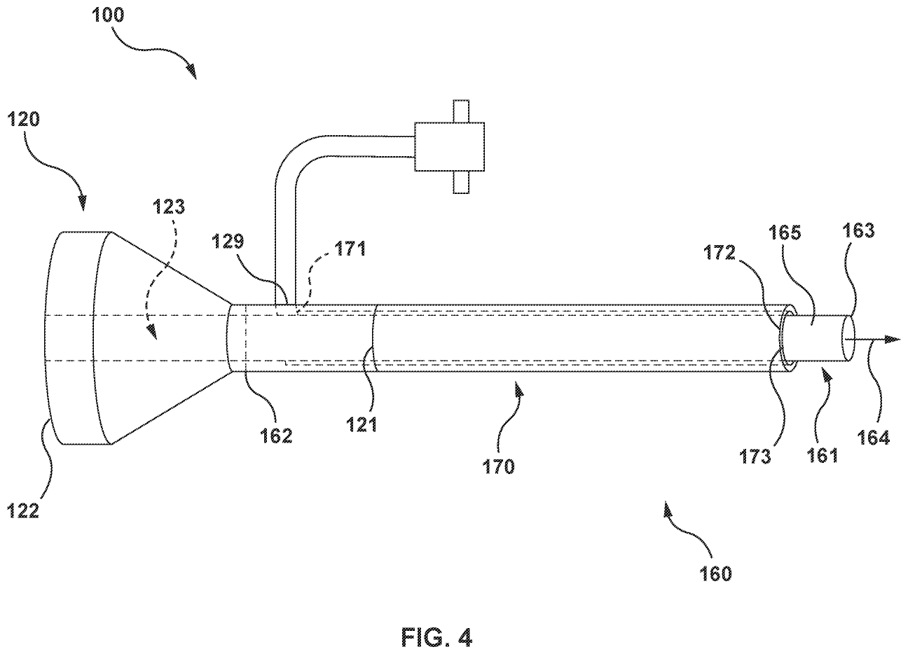

FIG. 4 shows an embodiment of an introducer 100 that includes an introducer sheath 160 having a porous outer layer 170 according with embodiments hereof. The introducer 100 includes an introducer hub 120 similar to the introducer hub 120 described above. Accordingly, the details of the introducer hub 120 will not be repeated.

The introducer sheath 160 includes a tube 161 having a proximal end 162 and a distal end 163, as shown in FIG. 4. The tube 161 includes a central lumen 164 through which a medical device such as a catheter delivery device may be delivered. The tube 161 may be is a thin, tubular structure. The proximal end 162 of the tube 161 may be disposed within and coupled to the introducer hub 120, as described above with respect to FIG. 1A. The central lumen 164 is in communication with the passageway 123 of the introducer hub 120 such that items such as medical devices passed through the passageway 123 of the introducer hub 120 enter the central lumen 164 of the tube 161. The tube 161 may be a polymer or nylon material, or other materials known to those skilled in the art suitable for the purposes described herein, or any combination thereof.

The porous outer layer 170 is disposed around an exterior surface 165 of the tube 161. The outer porous layer 170 includes a proximal end 171, a distal end 172, and a central lumen 173 extending from the proximal end 171 to the distal end 172. The central lumen 173 receives the tube 161 therein. In the embodiment shown, the proximal end 171 of the porous layer 170 is distal of the proximal end 162 of the tube 161 and distal of the port 129 of the introducer hub 129, but this is not meant to be limiting. Further, in the embodiment shown, the distal end 172 of the outer porous layer 170 is proximal of the distal end 163 of the tube 161, but this is also not mean to be limiting. An inner diameter of the outer porous layer 170 sized to contact the outer surface 165 of the tube 161. The outer porous layer 170 is coupled to the tube 161 via adhesive, fusion, mechanical fit, dip coating, spray coating, over moulding, etc. The outer porous layer 170 may be made from porous polymers including copolymers of styrene-divinylbenzene, sintered porous plastics such as Ultra-high molecular weight polyethylene (UHMWPE), high-density polyethylene (HDPE), polypropylene (PP), polytetrafluoroethylene (PTFE), and polyvinylidene fluoride (PVDF), Ethylene vinyl acetate (EVA), polyethersulfone (PES), polyurethane (PU) and polyethylene/polypropylene (PE/PP) co-polymer.

Similar to the embodiments above, a fluid such as a therapeutic agent may be injected through the port 129 to between the outer porous layer 170 and the outer surface 165 of the tube 161. As the therapeutic agent travels distally between the outer porous layer 170 and the tube 161, the therapeutic agent seeps through the outer porous layer 170 to the surrounding tissue. The vasodilators listed above may be the therapeutic agent used in this embodiment.

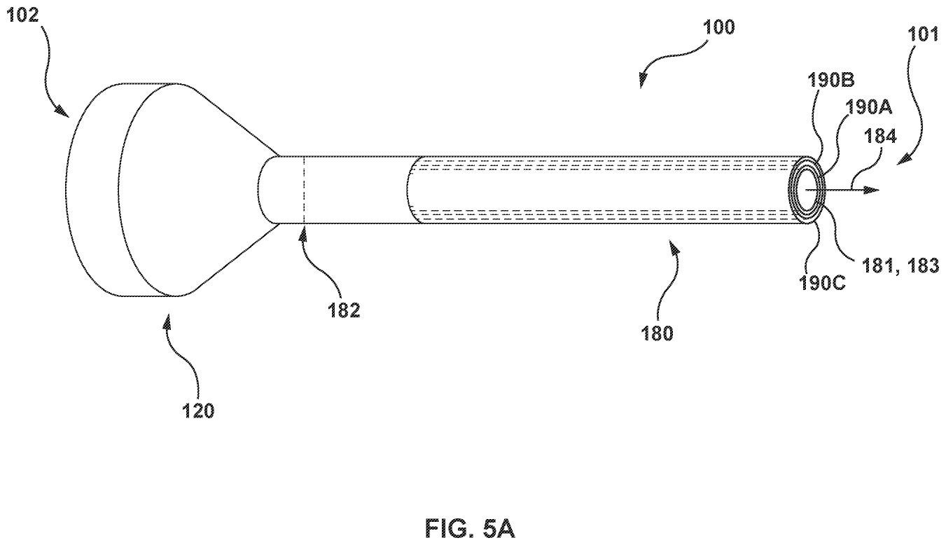

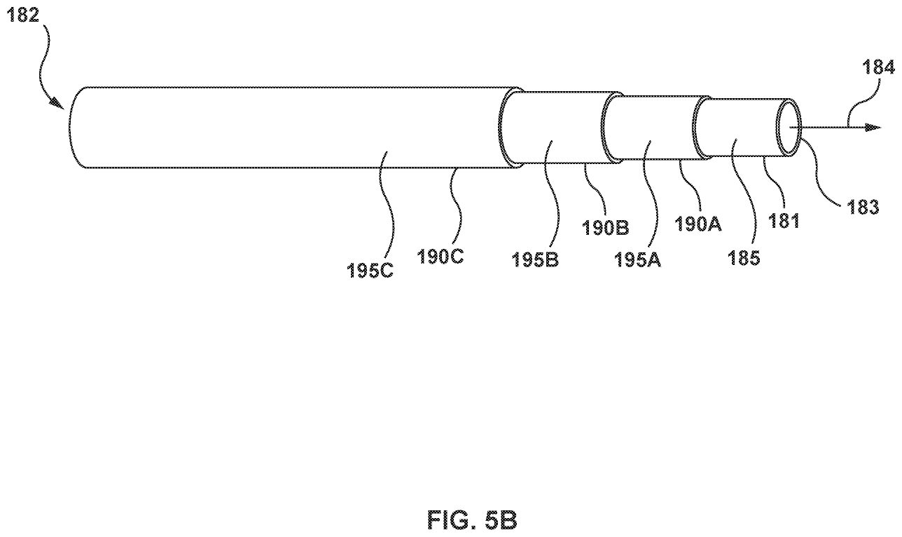

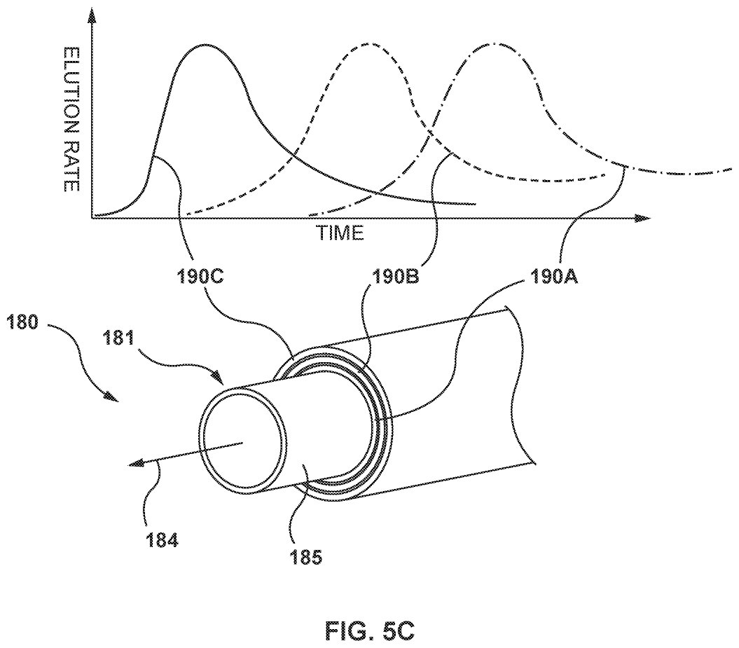

FIGS. 5A-5C show an embodiment of an introducer 100 including an introducer sheath 180 having at least one drug-coated outer layer 190 in accordance with embodiments hereof. The introducer 100 includes an introducer hub 120 similar to the introducer hub 120 described above. Accordingly, the details of the introducer hub 120 will not be repeated.

The introducer sheath 180 includes a tube 181 having a proximal end 182 and a distal end 183, as shown in FIG. 5A. The tube 181 includes a central lumen 184 through which a medical device such as a catheter delivery device may be delivered. The tube 181 may be a thin, tubular structure. The proximal end 182 of the tube 181 may be disposed within and coupled to the introducer hub 120, as described above with respect to FIG. 1A. The central lumen 184 is in communication with the passageway 123 of the introducer hub 120 such that items such as medical devices passed through the passageway 123 of the introducer hub 120 enter the central lumen 184 of the tube 181. The tube 181 may be a polymer or nylon material, or other materials known to those skilled in the art suitable for the purposes described herein, or any combination thereof.

The introducer sheath 180 further includes at least one drug-coated outer layer 190 disposed radially outward from an exterior surface 185 of the tube 181, as shown in FIGS. 5A-5C. The at least one drug-coated outer layer 190 is a drug-coated or drug-infused layer that is coupled to, deposited on, or otherwise disposed on the exterior surface of the tube 181 or other layers deposited thereon. The at least one drug-coated outer layer 190 includes a proximal end, a distal end, an exterior surface, and an interior surface that defines a central lumen. The central lumen of the drug-coated outer layer 190 is sized and shaped to conform to the exterior surface 185 of the tube 181, or the exterior surface of other outer layers, and extends from the proximal end to the distal end of the outer layer 190.

The embodiment shown and described herein includes exactly three drug-coated outer layers 190A, 190B, 190C disposed around the exterior surface 185 of the tube 181. However this is not meant to be limiting, as the introducer sheath 180 can include exactly one drug-coated outer layer 190 disposed around the tube 181, or exactly two drug-coated outer layers 190 disposed around the tube 181, or exactly four drug-coated outer layers 190 disposed around the tube 181, or exactly five drug-coated outer layers 190 disposed around the tube 181. In other embodiments, the introducer sheath 180 can include more than five drug-coated outer layers 190 disposed around the tube 181.

FIG. 5B shows an exploded view of an introducer sheath 110 having three coated outer layers 190A, 190B, 190C disposed thereon. As shown in FIG. 5B, a first or innermost drug-coated outer layer 190A is disposed on the exterior surface 185 of the tube 181. A second or middle drug-coated outer layer 190B is disposed on a first exterior surface 195A of the first drug-coated outer layer 190A. A third or outermost drug-coated outer layer 190C is disposed on a second exterior surface 195B of the second drug-coated outer layer 190B.

The first drug-coated outer layer 190A may have a thickness of about 0.001 to 0.1 mm. The second drug-coated outer layer 190B may have a thickness of about 0.001 to 0.1 mm. The third drug-coated outer layer 190C may have a thickness of about 0.001 to 0.1 mm. The drug-coated outer layers 190 may be a polymer coatings that include or encapsulate one or more therapeutic agents. The drug-coated outer layers may comprise one or more therapeutic agents dispersed within or encased by drug-polymer layers on the tube 181, which are eluted from tube 181 with controlled time delivery. For example, the therapeutic agent may be a therapeutic agent that causes dilation of the vessel into which the introducer 100 is inserted (i.e., a vasodilator). For example, and not by way of limitation, the therapeutic agent may be selected from the group consisting of cyclandelate, isoxsuprine, papaverine, dipyridamole, isosorbide dinitrate, phentolamine, nicotinyl alcohol, co-dergocrine, nicotinic acid, glycerol trinitrate, pentaerythritol tetranitrate, xanthanol, and other vasodilators known to those skilled in the art.

In some embodiments, the coated outer layers 190A, 190B, 190C may extend from the distal end 121 of the introducer hub 120 to the distal end 183 of the tube 181. In other embodiments, the coated outer layers 190A, 190B, 190C need not extend the entire length between the distal end 121 of the introducer hub 120 and the distal end 183 of the tube 181.

In embodiments, the drug-coated outer layers 190A, 190B, and 190C may be configured to release the vasodilator at different times while the introducer sheath 180 is disposed in the body. For example, and not by way of limitation, the drug-coated outer layers 190A, 190B, 190C may be configured to release the vasodilator upon exposure to blood or other fluids in the vessel. Thus, the outermost drug-coated outer layer 190C would elute the vasodilator first, then the middle layer 190B, then the inner layer 190C. FIG. 5C shows an example of elution from the drug-coated outer layers 190A, 190B, 190C over time. The elution rates and time release of the drug-coated outer layers may be varied by varying factors such as, but not limited to, the thickness of the drug-coated outer layers 190, the material of the drug-coated outer layers, adding non-drug-coated layers between the drug-coated outer layers or outside of the outermost drug-coated outer layer, and/or other factors known to those skilled in the art.

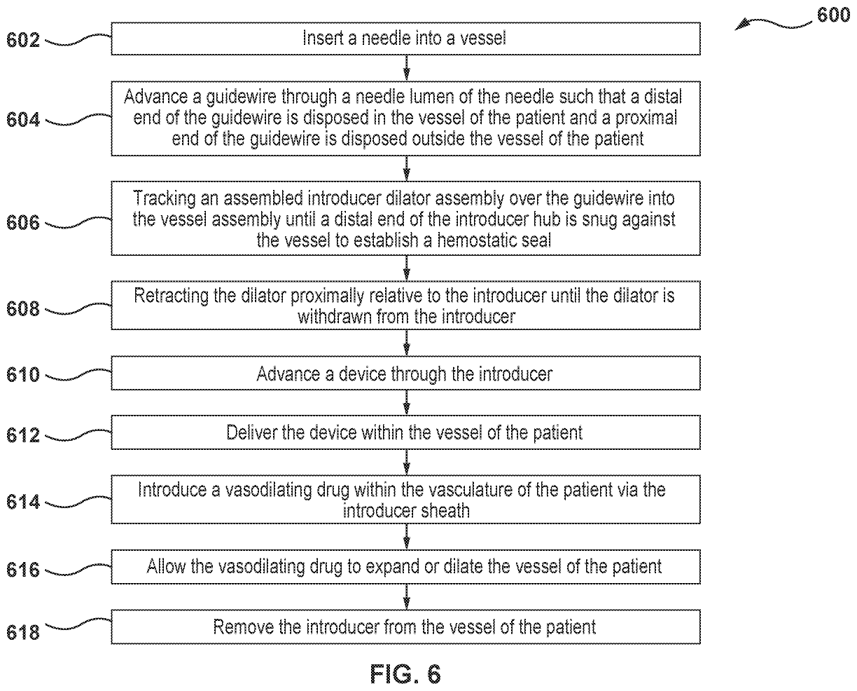

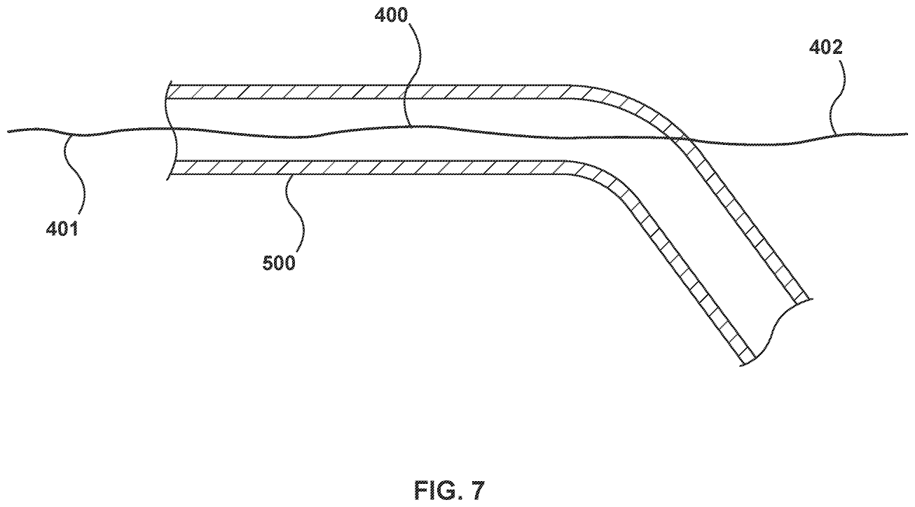

FIG. 6 is a block diagram of a method 600 for accessing the vasculature 500, delivering a device to a desired site within the vasculature 500, and removing the introducer dilator assembly 300 from the vasculature 500. The device (not shown) can be, for example, a catheter delivery system for a delivering and deploying a heart valve prosthesis. In a step 602 of the method 600, a needle (not shown) may be inserted through a wall of the vasculature 500, wherein the needle includes a needle lumen. In another step 604 of the method 600, with the needle having gained access to the vessel, a guidewire 400 is advanced through the needle lumen and extends through a distal end of the needle lumen. Thus, a distal end 401 of the guidewire 400 is disposed in the vessel 500 of the patient and a proximal end 402 of the guidewire 400 is disposed outside of the patient, as shown in FIG. 7.

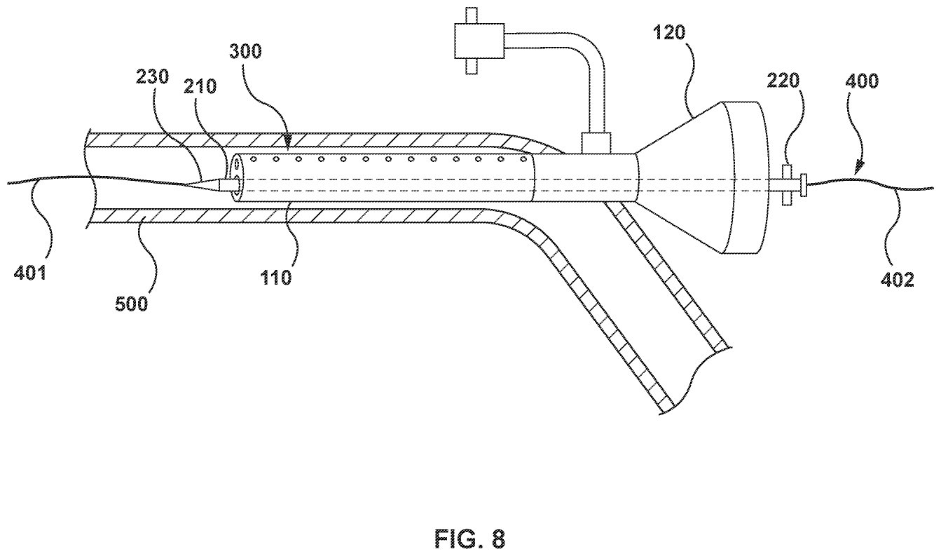

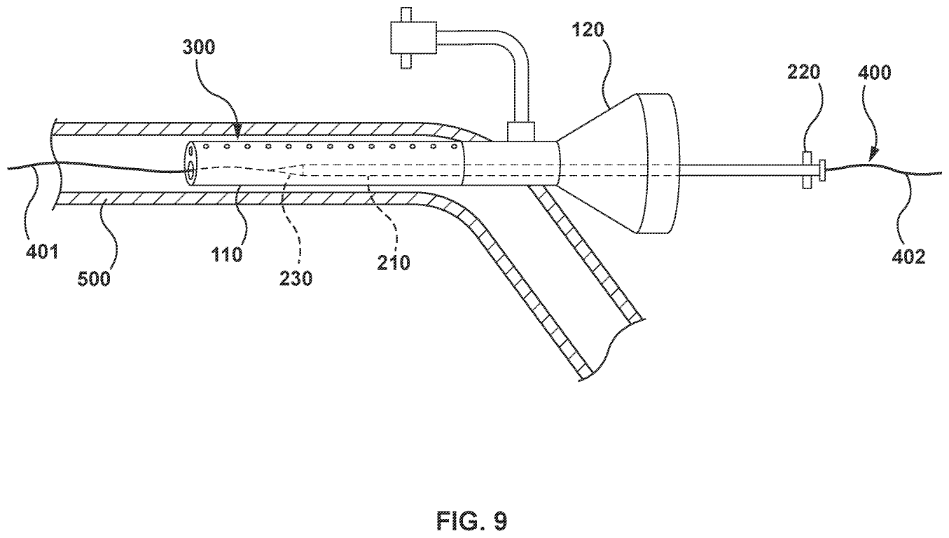

In a step 606 of the method 600, the proximal end 402 of the guidewire 400 is inserted into a distal end of the central lumen 213 of the dilator shaft 210 of the dilator 200, with the dilator 200 inserted through the introducer 100 to create the introducer dilator assembly 300. The introducer dilator assembly 300 is then distally advanced over the guidewire 400 into the vessel 500 of the patient until the distal end 121 of the introducer hub 120 is snug against the vessel 500, as shown in FIG. 8. The proximal end 122 of the introducer hub 120 and the dilator hub 220 remain outside of the vasculature 500 of the patient. In a step 608 of the method 600, the dilator 200 is retracted proximally through the primary lumen 113 of the introducer sheath 110 of the introducer 100 until the dilator 200 is withdrawn from the introducer 100, as shown in FIG. 9. Once the dilator 200 is removed, the introducer 100 and the guidewire 400 remain within the vasculature 500 of the patient.

In a next step 610 of the method 600, the device (not shown) may be advanced through the introducer 100 over the guidewire 400 into the proximal end 122 of the introducer hub 120 and advanced distally therethrough. As the device advances distally through the central lumen 113 of the introducer sheath 110, the introducer sheath 110 may expand from a first diameter to a second diameter larger than the first diameter if the introducer sheath is an expandable introducer sheath. In a next step 612 of the method 600, the device advances distally through the central lumen 113 and exits through the distal end 111 of the introducer sheath 110, wherein the device can continue to be tracked over the guidewire 400 until it reaches a desired location within the vasculature 600 of the patient.

In a next step 614 of the method 600, a therapeutic agent is released from the introducer sheath 110. As explained above, the therapeutic agent is a vasodilating drug. The therapeutic agent may be released from the introducer sheath depending on the type of introducer sheath (e.g. 110, 160, 180) used, as described above. The step 614 of releasing the therapeutic agent may happen at any time after the introducer sheath 110, 160, 180 has been introduced into the vasculature 500. Therefore, in an example, the therapeutic agent may be administered prior to the medical device being delivered through the introducer sheath, during advancement of the medical device through the introducer sheath, after the medical device is delivered through the introducer sheath, during removal of the delivery device through the introducer, and/or during removal of the introducer from the vasculature. In some embodiments, the therapeutic agent is administered during the entire procedure, such as through the time release drug-coated outer layers described above. In other embodiments, the therapeutic agent is administered just prior to and/or during removal of the introducer sheath from the vasculature.

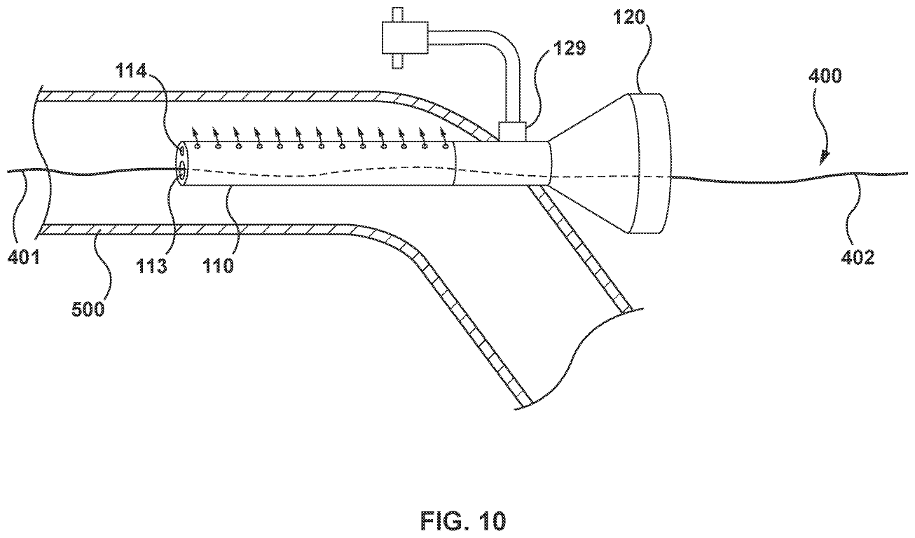

In a step 616 of the method 600, when the vasodilating drug is delivered within the vasculature 500 of the patient, the vasodilating drug causes the vasculature 500 of the patient to expand or dilate such that the interior wall of the vessel 500 is spaced radially outward from the exterior surface of the introducer sheath, as shown in FIG. 10. In a final step 618 of the method 600, the introducer 100 is retracted proximally until it is removed from the vasculature 500 of the patient. The dilation of the vessel 600 allows the introducer 100 to be removed from the vasculature 600 without the introducer sheath sticking to or pulling on the interior wall of the vasculature 600 during removal.

Those skilled in the art will recognize that other steps may be included and that the order in which the steps are described are not necessarily limiting. It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

What is claimed is:

1. An introducer comprising:
an introducer hub; and
an introducer sheath defining a primary lumen configured to receive a medical device therethrough,
wherein a vasodilating therapeutic agent is configured to be released from the introducer sheath to expand a vessel into which the introducer sheath is inserted.

2. The introducer of claim 1, wherein the introducer sheath further includes an infusion lumen adjacent to the primary lumen and a plurality of ports extending from an exterior surface of the introducer sheath to the infusion lumen, wherein the introducer sheath is configured to receive the vasodilating therapeutic agent in the infusion lumen and to release the vasodilating drug through the plurality of ports.

3. The introducer of claim 2, wherein the introducer sheath includes exactly one infusion lumen, or exactly two infusion lumens, or exactly three infusion lumens, or four or more infusion lumens.

4. The introducer of claim 2, wherein the infusion lumen is an annular lumen surrounding the primary lumen.

5. The introducer of claim 2, wherein the plurality of ports extend from adjacent a distal end of the introducer hub to adjacent a distal end of the introducer sheath, or wherein the plurality of ports extend along only a distal portion of the introducer sheath, or wherein the plurality of ports extend along only a proximal portion of the sheath.

6. The introducer of claim 2, wherein the plurality of ports increase in size in a distal direction along the longitudinal length of the introducer sheath, or wherein the plurality of ports are uniform in size.

7. The introducer of claim 1, wherein the introducer sheath includes a tube having the primary lumen disposed therethrough and an outer porous layer covering an exterior surface of the tube.

8. The introducer of claim 7, wherein the outer porous layer comprises a copolymers of styrene-divinylbenzene or a sintered porous plastic.

9. The introducer of claim 7, wherein the outer porous layer extends from a proximal end of the tube to a distal end of the tube, or wherein the outer porous layer extends over only a proximal portion of the introducer sheath.

10. The introducer of claim 1, wherein the introducer sheath comprises a tube having the primary lumen disposed therethrough and at least one drug-coated layer disposed on an exterior surface of the tube, wherein the at least one drug-coated layer includes the vasodilating therapeutic agent.

11. The introducer of claim 10, wherein the at least one drug-coated layer is configured to elute the vasodilating therapeutic agent upon contact with bodily fluids in the vessel.

12. The introducer of claim 15, wherein the at least one drug-coated layer comprises a plurality of drug-coating layers configured to elute the vasodilating therapeutic agent over time.

13. The introducer of claim 12, wherein a first coated outer layer releases a drug over a first period of time, a second coated outer layer releases a drug over a second period of time that occurs after the first period of time, and a third coated outer layer releases a drug over a third period of time that occurs after the second period of time, and
wherein the first period of time, the second period of time, and the third period of time overlap with one another or the first period of time, the second period of time, and the third period of time do not overlap with one another.

14. The introducer of claim 10, wherein the at least one drug-coated layer extends over from a distal end of the introducer to a distal end of the tube, or wherein the at least one drug-coated layer extends over only a proximal portion of the tube, or wherein the at least one drug-coated layer extends over only a distal portion of the tube.

15. A method comprising:
inserting an introducer into a vessel of a patient such that an introducer sheath of the introducer is disposed within the vessel and an introducer hub of the introducer is disposed outside of the patient;
advancing a device distally through the introducer sheath;
introducing a vasodilating therapeutic agent within the vessel through the introducer such that the vasodilating therapeutic agent dilates the vessel; and
removing the introducer sheath from the vessel after the vessel is dilated.

16. The method of claim 15, further comprising:
prior to removing the introducer from the vessel, removing the device from the vessel through the introducer sheath.

17. The method of claim 16, wherein the vasodilating therapeutic agent is introduced prior to removing the device from the vessel.

18. The method of claim 16, wherein the vasodilating therapeutic agent is introduced after removing the device from the vessel.

19. The method of claim 15, wherein the introducer comprises any introducer comprises:
an introducer hub; and
an introducer sheath defining a primary lumen configured to receive a medical device therethrough,
wherein introducing the vasodilating therapeutic agent within the vessel through the introducer comprises introducing through the introducer sheath.

20. The method of claim 19,
wherein the introducer sheath further includes an infusion lumen adjacent to the primary lumen and a plurality of ports extending from an exterior surface of the introducer sheath to the infusion lumen, wherein the introducer sheath is configured to receive the vasodilating therapeutic agent in the infusion lumen and to release the vasodilating drug through the plurality of ports; or
wherein the introducer sheath includes a tube having the primary lumen disposed therethrough and an outer porous layer covering an exterior surface of the tube; or
wherein the introducer sheath comprises a tube having the primary lumen disposed therethrough and at least one drug-coated layer disposed on an exterior surface of the tube, wherein the at least one drug-coated layer includes the vasodilating therapeutic agent.
